(19) United States
(12) Patent Application Publication
(10) Pub. No.: US 2021/0393758 A1
(43) Pub. Date: Dec. 23, 2021

(54) HYALURONIC ACID AS A NATURAL ADJUVANT FOR PROTEIN AND PEPTIDE-BASED VACCINES

(71) Applicant: ISTITUTO ONCOLOGICO VENETO IOV-IRCCS, Padova (IT)

(72) Inventors: Antonio ROSATO, Padova (IT); Isabella Monia MONTAGNER, Padova (IT); Debora CARPANESE, Padova (IT); Anna DALLA PIETA', Padova (IT)

(21) Appl. No.: 17/288,656

(22) PCT Filed: Oct. 24, 2019
(86) PCT No.: PCT/IB2019/059122

§ 371 Date: Apr. 26, 2021

(30) Foreign Application Priority Data
Oct. 26, 2018 (IT) 102018000009836

(51) Int. Cl. A61K 39/00; A61K 39/39

(52) U.S. Cl. CPC A61K 39/001106; A61K 2039/55583; A61K 39/39

(57) ABSTRACT

Preparation of hyaluronic acid based vaccines and a prophylactic or therapeutic method includes vaccinating a patient with hyaluronic acid.

The present invention finds application in the field of medicine and, in particular, for the preparation of vaccines.

Hyaluronic acid (HA) is a natural polysaccharide with a linear structure of repeating disaccharide units composed of D-glucuronic acid and N-acetyl-D-glucosamine. HA displays a proven clinical safety, as it has been widely used for long-acting delivery applications of nucleotide, peptide, and protein therapeutics.

### SUMMARY OF THE INVENTION

The inventors of the present patent application have found that hyaluronic acid polymers characterized by a specific molecular weight range can be used for the development and for the preparation of vaccines, being endowed with inherent strong adjuvanticity.

### BRIEF DESCRIPTION OF THE FIGURES

FIG. 1: Balb/c mice immunized i.m. with the standard immunization schedule (priming+I and II boost protocol) with OVA (10 μg of antigen) conjugated with HA at different molecular weight (15, 50, 200, and 500 kDa). ELISA test for the total IgG content of sera collected at day 30. Data are expressed as the Absorbance at different sera dilutions.

FIG. 2: Balb/c mice immunized i.m. with different antigens (10 μg of antigen, standard immunization schedule) conjugated to 200 kDa HA, emulsified in Alum or injected alone: ELISA test for the total IgG content of sera collected at day 30. Data are expressed as reported in FIG. 1.

FIG. 3: Balb/c mice immunized i.m. with a single priming of OVA: ELISA test for the total IgG content of sera collected at day 30. Data are expressed as reported in FIG. 1.

FIG. 4: Balb/c mice immunized i.m. (standard immunization schedule) with different amounts (0.1, 1, and 10 μg) of HA-conjugated OVA: ELISA test for the total IgG content of sera (1:50 dilution) collected at day 14, 21, and 30. Data are expressed as Absorbance at the different time points.

FIG. 5: Transgenic Balb-neuT mice vaccinated i.m. with rHER2/neu-HA or the antigen emulsified in Alum (10 μg, standard immunization schedule): ELISA test for the total IgG content of sera collected at day 30. Data are expressed as reported in FIG. 1.

FIG. 6: Balb/c mice immunized i.m. with 10 μg of HA-OVA, OVA emulsified with different adjuvants (LPS, Chitosan, Quil-A, Alum, CFA/IFA, Addavax, ISA51, and ISA720), or OVA alone. ELISA test for the total IgG content of sera (1:50 dilution) collected at day 30. A) Single priming protocol and B) priming+I and II boost protocol. Data are expressed as Absorbance for each experimental condition.

FIG. 7: Balb/c mice vaccinated i.m. with 10 μg of rHER2/neu-HA or rHER2/neu emulsified in Alum or the protein alone (standard immunization schedule): Complement-Dependent Cytotoxicity assay. ⁵¹Cr-labeled 3T3/NKB (rHER2/neu-positive) cells were incubated with sera collected from day 0 to day 360, and then with rabbit complement. Data report the specific lysis obtained at the indicated time points.

FIG. 8: Prophylactic vaccination: effects on tumor growth. Balb/c mice were vaccinated i.m. with 10 μg of rHER2/neu-HA or rHER2/neu emulsified in Alum (standard immunization schedule). At day 30 (in graph: day 0), they were challenged with 1×10⁵ rHER2-expressing TUBO cells injected into the mammary fat pad. Control group was represented by not-vaccinated mice that received tumor challenge. From the day of tumor challenge, tumor growth was monitored and reported in the graph as tumor volume (mm³). Mice vaccinated with the bioconjugate resulted to be completely protected from tumor growth.

FIG. 9: Vaccination of transgenic Balb-neuT mice: effects on tumor multiplicity. Balb-neuT mice were vaccinated i.m. with 10 μg of rHER2/neu-HA or rHER2/neu emulsified in Alum (standard vaccination schedule). Control group was represented by untreated mice. All animals were monitored for tumor appearance at any distinct mammary gland. Data are reported as tumor multiplicity (cumulative number of neoplastic mammary glands per mouse in each group) per week of age.

FIG. 10: Therapeutic vaccination: effects on tumor growth. Balb/c mice were challenged with 1×10⁵ TUBO cells injected into the mammary fat pad, and when all tumors were detectable (called day 0), mice were vaccinated i.m. at days 0, 7, and 14 with 10 μg of rHER2/neu-HA or rHER2/neu emulsified in Alum. Control group is represented by mice challenged with tumor but not vaccinated. From the day of tumor challenge, tumor growth was monitored and reported in the graph as tumor volume (mm³).

FIG. 11: Transgenic Balb-neuT mice: ⁵¹Cr-release assay. At the day of sacrifice (when at least one tumor reached a volume of 1500 mm³), spleens were harvested and restimulated in vitro with rHER2/neu-expressing 3T3/NKB and TUBO cells or rHER2/neu-negative NIH/3T3 cells (TARGETS). After 5 days, the restimulated splenocytes (EFFECTORS) were incubated with ⁵¹Cr-labeled TARGET cells at different effector/target ratios. Figure reports the specific target cell lysis obtained at an effector/target ratio of 100:1 only for mice vaccinated with rHER2/neu-HA, since no lysis was obtained for Balb-neuT mice immunized with rHER2/neu emulsified in Alum.

### DETAILED DESCRIPTION OF THE INVENTION

According to a first object of the invention, hyaluronic acid is described for medical use in a prophylactic or therapeutic method in vaccination protocols.

According to an alternative embodiment, hyaluronic acid is described for medical use in a prophylactic or therapeutic method as a vaccine.

In particular, hyaluronic acid is used as an immunological adjuvant.

For the purposes of the present invention, an immunological adjuvant is defined as a substance which is added to the vaccine formulation in order to modulate, prolong and/or enhance the specific immune response against the antigen/s contained in the vaccine; an adjuvant modifies or increases the effects of a vaccine by stimulating the immune system to respond to the vaccine more vigorously, and thus providing increased immunity.

For the purposes of the present invention, the medical use encompasses the veterinary application; accordingly, the present invention may find application for human and also for animals, particularly for pets and for livestock.

According to a preferred aspect of the present invention, hyaluronic acid has a molecular weight of about 10-500 KDa, more preferably of about 15-200 KDa and even more preferably of about 50-200 KDa.

For the purposes of the present invention, hyaluronic acid is used in conjugates that comprise hyaluronic acid and an antigen; in other words, hyaluronic acid is used in the form of a conjugate comprising an antigen.

In a preferred aspect of the invention, hyaluronic acid is chemically bound to an antigen.

This bond is of the covalent type and preferably is obtained by means of appropriate linkers.

Linkers may be suitably selected by the person skilled in the art among those used for the conjugation of biological moieties; for instance, the U.S. Pat. No. 9,034,624 discloses possible and suitable linkers.

For the purposes of the present invention, hyaluronic acid is not a vehicle or a carrier for an antigen; rather, the hyaluronic acid as such is endowed with adjuvant properties.

In a preferred embodiment, the potentiality of hyaluronic acid is similar or even superior to already known and widely used adjuvants like: Alum, Quil-A, Addavax, Complete or Incomplete Freund's Adjuvant (CFA/IFA), Chitosan, LPS and Montanide (ISA 51 and ISA 720).

According to an object of the present invention, the antigens to which hyaluronic acid can be bound are proteins or peptide-based antigens.

According to an even preferred embodiment of the invention, the antigens linked to hyaluronic acid are:

OVA (ovalbumin) as an example of protein antigen (L1 surface protein of human papilloma virus, HPV; HBsAg, Hepatitis B surface antigen; PARK2, Parkinson Disease Protein 2 Human; Meningococcal group B; Serpin; Amyloid Beta Precursor Protein for Alzheimer's disease)

RNAse as an example of antigen with enzymatic activity (SOD, Superoxide dismutase)

hGH (human Growth Hormone) as an example of antigen with hormone activity (TSH, Thyroid Stimulating Hormone; hCG, human Chorionic Gonadotropin)

TT (Tetanus Toxoid) as an example of bacterial products and toxoids (Anthrax Toxin; Diphtheria Toxoid; Botulinum Toxoid; Pertussis Toxoid).

Haemaglutinin from H5N1 (Influenza virus) as an example of pathogen antigens (neuraminidase (NA) of influenza viruses; p17 HIV matrix protein, human immunodeficiency virus (HIV); gp120 HIV membrane protein; Glycoprotein (G-Protein) of Rabies virus; MPT64 Mycobacterium tuberculosis-derived protein; B and T epitopes for Group A Streptococci; Autoclaved S. mansoni antigen (ASMA) from Schistosoma mansoni; ES-62 from Acanthocheilonema viteae; F and G proteins of respiratory syncytial virus (RSV); circumsporozoite (CS) protein of Plasmodium parasite of Malaria; herpes simplex viruses; Pertactin, filamentous haemagglutinin (FHA) and Fimbriae of Pertussis; all antigens from viruses (i.e., Chikungunya, Cytomegalovirus, Dengue, Ebola, EBV, Encephalitis, Feline Leukemia Virus, Hantavirus, Hepatitis A, B, C, D and E, Herpes viruses, HIV, HTLV, Influenza, Lassa, Measles, Mumps, Norovirus, Papilloma virus, Parvovirus, Rubella, SARS, Varicella, West Nile or Zika), fungi (i.e., Aspergillus and Saccharomyces), parassites (i.e., Plasmodium falciparum, Plasmodium vivax, Echinococcus granulosus, Leishmania, Malaria, Schistosoma japonicum, Toxoplasma gondii, Trichomonas vaginalis, Trypanosoma cruzi) and bacteria (i.e., Aeromonas, Borrelia, Bordetella pertussis, Campylobacter jejuni, Candida albicans, Chlamydia pneumonia, Chlamydia trachomatis, Clostridium, Corynebacterium diphtheriae, Haemophilus influenzae, Legionella pneumophila, Leptospira biflexa, Listeria monocytogenes, Mycobacterium, Pseudomonas aeruginosa, Salmonella, Streptococcus pneumoniae, Clostridium tetanis, Treponema pallidum).

HER2/neu (human epidermal growth factor receptor 2) as an example of tumor antigen (Carcinoembryonic antigen (CEA), Colorectal carcinoma; Immature laminin receptor, Renal cell carcinoma; TAG-72, Prostate carcinoma; E6 and E7 HPV-derived oncoproteins, Human papilloma virus; LMP1, LMP2, EBNA-1 and EBNA-2 EBV-derived oncoproteins, Epstein Barr Virus; HBpol HBV-derived oncoprotein, Hepatitis B virus; NS5 HCV-derived oncoprotein, Hepatitis C virus; BING-4, Melanoma; Calcium-activated chloride channel 2, Lung carcinoma; Cyclin-B1; EGFR epidermal growth factor receptor; 9D7, Renal cell carcinoma; Ep-CAM, Breast carcinoma; EphA3; Telomerase; Mesothelin; Ductal pancreatic cancer; SAP-1, Colorectal carcinoma; Survivin; BAGE family; CAGE family; GAGE family; MAGE family; SAGE family; XAGE family; NY-ESO-1/LAGE-1; PRAME; SSX-2; Melan-A/MART-1, Melanoma; Gp100/pmel17, Melanoma; Tyrosinase, Melanoma; TRP-1/-2, Melanoma; MC1R, Melanoma; Prostate-specific antigen, Prostate cancer; mutated β-catenin; mutated BRCA1/2, breast cancer antigen; mutated CDK4; mutated CML66, Chronic myelogenous leukemia; mutated Fibronectin; mutated MART-2, melanoma; mutated p53; mutated Ras; mutated TGF-βRII, colorectal carcinoma; MUC1).

Vκ3-20 (non-Hodgkin's lymphoma light chain idiotype) as an example of idotypic model antigen (i.e., Ig, TCR B, T leukemia, lymphoma, myeloma).

HER2/neu peptide as an example of peptides derived from the above mentioned classes of antigens.

In a particular embodiment, the hyaluronic acid of the present invention fosters the cell-mediated immune response and enables the generation of specific cytotoxic T-cell immunity.

In a further particular embodiment, the hyaluronic acid of the present invention breaks the immunologic tolerance against a self-antigen.

For the purposes of the present invention, a vaccination protocol is a standard immunization schedule.

In an embodiment of the present invention, the immunization schedule comprises a single injection.

In a preferred embodiment, it comprises a second boost at day 14 (the first boost being a day 0) and,

in an even preferred embodiment, a third boost at day 21.

For the purposes of the present application the immunization schedule may comprise as many administrations as required by the circumstances, considering the kind of antigen and the subject to whom the vaccine is to be administered.

The hyaluronic acid conjugates here disclosed are used in an amount of antigen which is 0.1 μg, preferably is 1 μg and even more preferably is 10 μg.

The person skilled in the art will be able to determine the amount of antigen to be administered to a subject, human or animal, in each administration, according to standard parameters, such as for example: the type of antigen, the sex, age, weight, body surface of the subject, etc.

According to an aspect of the present invention, additional routes of administration can be represented by the intravenous (i.v.), intraperitoneal (i.p.), subcutaneous (s.c), intranasal, intravitreal or rectal routes, beside the classical intramuscular (i.m.) administration.

In particular, both the intravenous and intramuscular inoculation is capable of eliciting the production of antigen-specific IgG which are still detectable after one year after the first inoculation, preferably after 5 years after the first inoculation, more preferably after 10 years after the first inoculation and in even more preferably for the entire life of the subject.

In a particular embodiment of the invention, after administration to human or animal, specific subclasses of IgG are detectable.

According to an aspect of the present invention, the disclosed hyaluronic acid conjugates showed to elicit not only the production of IgG1 (associated with Th2 response) but also of IgG2a and IgG2b subclasses, which are normally linked to a Th1-skewed immune response in mice.

Thus, the hyaluronic acid conjugates of the invention stimulate a balanced Th1/Th2 response that is a critical step to be considered during vaccine formulation in order to activate both humoral and cell-mediated pathways of the immune system.

In a preferred embodiment, the hyaluronic acid conjugates do not produce cell infiltration caused by inflammation after the inoculation; particularly, they do not induce nor cause inflammation at the inoculum site.

According to a second object of the invention hyaluronic acid conjugates are disclosed for use in a method for enhancing the immunogenicity of an antigen in a prophylactic or therapeutic method.

In a preferred embodiment, the hyaluronic acid conjugates are capable of inducing a stronger humoral response and of increasing the total production of IgG.

In a still preferred embodiment, the disclosed conjugates are capable of enhancing the production of IgG1, IgG2a and IgG2b subclasses after administration in mice; corresponding IgG subclasses production is enhanced in human or animals.

In an even still preferred embodiment, the IgG are still detectable 1 year after the first injection (in mice) and, particularly, there are detectable IgG2a and IgG2b. After administration in human or animal corresponding sub-classes of IgG may be detected after one year after the first inoculation, preferably after 5 years after the first inoculation, more preferably after 10 years after the first inoculation and in even more preferably for the entire life of the subject.

According to a third object, the present invention discloses hyaluronic acid conjugates for use in a prophylactic or therapeutic method for eliciting the immune response against oncoviral and self-antigens in a patient.

In particular, the term “oncoviral antigens” shall be intended as tumor antigens produced by oncogenic viruses.

In a preferred embodiment, said oncoviral antigens are: E6 and E7 HPV-derived oncoproteins, Human Papilloma Virus; LMP1, LMP2, EBNA-1 and EBNA-2 EBV-derived oncoproteins, Epstein Barr Virus; HBpol HBV-derived oncoprotein, Hepatitis B Virus; NS5 HCV-derived oncoprotein, Hepatitis C Virus.

In particular, said immune response against a self-antigen is elicited under the circumstances wherein the expression of the self-antigen is connected to a neoplasia.

For the purposes of the present application, self-antigens are intended to be those antigens that are naturally developed and naturally present in the body; accordingly, said antigens are tolerated by the immune system, which does not normally develop an immunologic response against them.

In one embodiment, the self-antigens are also over-expressed and over-expression is connected to a neoplasia.

“Over-expression” shall be intended as an anomalous increment in the expression of an antigen as compared with the normal expression by cells in healthy tissues.

The term “naturally” shall be intended as those conditions wherein immunologic pathologies, i.e. wherein the immunologic system is altered, are absent.

In a preferred embodiment, said antigens are: human epidermal growth factor receptor 2 (HER2/neu), breast cancer; BING-4, Melanoma; Calcium-activated chloride channel 2, Lung carcinoma; Cyclin-B1; EGFR epidermal growth factor receptor; 9D7, Renal cell carcinoma; Ep-CAM, Breast carcinoma; EphA3; Telomerase; Mesothelin, ductal pancreatic cancer; SAP-1, Colorectal carcinoma; Survivin; MUC1; Melan-A/MART-1, Melanoma; Gp100/pmel17, Melanoma; Tyrosinase, Melanoma; TRP-1/-2, Melanoma; MC1R, Melanoma; Prostate-specific antigen, Prostate cancer.

In another embodiment, the self-antigens are over-expressed and mutated and over-expression and mutation are connected to a neoplasia.

In still another embodiment, the self-antigens are mutated and mutation is connected to a neoplasia.

In a preferred embodiment, said antigens are: mutated β-catenin; mutated BRCA1/2, breast cancer antigen; mutated CDK4; mutated CML66, Chronic myelogenous leukemia; mutated Fibronectin; mutated MART-2, melanoma; mutated p53; mutated Ras; mutated TGF-βRII, colorectal carcinoma.

In yet another embodiment, the self-antigens are fetal antigens and the expression of said fetal antigen is connected to a neoplasia.

For the purposes of the present invention, fetal antigens are those antigens which are typically present only during fetal development, but may be found in adults with certain kinds of cancer and are connected to a neoplasia.

In a preferred embodiment, said fetal antigens are: Carcinoembryonic antigen (CEA), Colorectal carcinoma; Immature laminin receptor, Renal cell carcinoma; TAG-72, Prostate carcinoma.

In yet another embodiment, the self-antigens are cancer testis antigens (CTA) and the expression of said CTA is connected to a neoplasia.

Cancer/testis (CT) antigens are a category of antigens with normal expression restricted to male germ cells in the testis but not in adult somatic tissues; in case of neoplasia, CT antigens are abnormally expressed in somatic tissue.

In a preferred embodiment, said CTA antigens are: BAGE family; CAGE family; GAGE family; MAGE family; SAGE family; XAGE family; NY-ESO-1/LAGE-1; PRAME; SSX-2.

In other words, according to the present invention, the prophylactic or therapeutic method is performed on a patient who presents oncogenic self-antigens (or antigens whose presence is connected to a neoplasia).

In a preferred embodiment of the invention, the disclosed hyaluronic acid conjugates are capable of eliciting an immune response against the self-antigen HER2/neu oncogene.

In a particular embodiment, said effect is produced after a first dose of vaccine, preferably after a second dose of vaccine and more preferably after a third or a further dose of vaccine.

According to a further object of the invention, hyaluronic acid conjugates are disclosed for use in a therapeutic or prophylactic method for eliciting a cell-mediated immune response in a vaccination protocol.

According to another object of the invention, it is disclosed hyaluronic acid for use in a therapeutic or prophylactic method in a vaccination protocol for stimulating the complement-dependent cytotoxicity in a patient.

In particular, for the purposes of the present invention, the subject has been vaccinated with a conjugate comprising hyaluronic acid and an antigen.

More in particular, hyaluronic acid is capable of inducing antibodies that activate Complement and lead to the lysis of cells expressing on their surface the same antigen, which has been used for the vaccination.

Moreover, hyaluronic acid conjugates are capable of eliciting cytotoxic T lymphocytes that lyse target cells expressing the antigen used for vaccination.

As per another object of the present invention, the disclosed hyaluronic acid conjugates are disclosed for use in a method for the prophylaxis, the delaying and the therapy of pre-neoplastic lesions.

“Pre-neoplastic lesions” shall be intended as a precancerous condition in which there are detectable lesions involving abnormal cells which are associated with an increased risk of developing into cancer.

For the purposes of the present invention, delaying shall be intended as the capacity of the conjugates to delay the development and/or to delay the growth of a neoplasia with respect to a control group; in particular, it means that a longer period of time is required before the neoplasia reaches the same volume of a control group at a time.

According to a specific embodiment of the invention, the conjugates of the invention comprising hyaluronic acid and HER2/neu proved to delay the development and the growth of the neoplasia.

For the purposes of the present invention, prevention shall be intended as the capacity of the hyaluronic acid conjugates to induce the development of a smaller neoplasia with respect to a control sample or to prevent at all the development of a neoplasia.

In a still further embodiment of the present invention, hyaluronic acid is disclosed for use in a method for the treatment of oncologic pathologies, wherein the growth of the neoplasia is delayed.

In such a preferred embodiment, the hyaluronic acid is capable of delaying the development and growth of an already existing neoplasia.

In a further embodiment of the invention, the hyaluronic acid conjugates are disclosed for use in a method for the treatment of oncologic pathologies, wherein the neoplasia undergoes regression.

For the present purposes, regression is intended as a decrease in the mass of the neoplasia and includes the complete regression of the neoplasia.

In particular, said method comprises the vaccination of a patient with an already existing neoplasia.

According to an even further aspect of the invention, all the above disclosed objects find application in the veterinary field for the treatment of animals.

In particular, animals include both pets and livestock.

The disclosed conjugates of hyaluronic acid with the antigens do represent further objects of the present invention.

In particular, hyaluronic acid has a molecular weight of from about 10 to 500 KDa, more preferably of from about 15-200 KDa and even more preferably of from about 50-200 KDa.

In a preferred embodiment, the hyaluronic acid and the antigen are chemically bound.

As above disclosed, the antigen may be a protein-based or a peptide-base antigen.

In a preferred embodiment, the antigen is selected in the group comprising: OVA (ovalbumin), BSA (Bovine Serum Albumin), RNAse, Haemaglutinin from H5N1 (Influenza virus), hGH (human Growth Hormone), TT (Tetanus Toxoid), HER2/neu (Human Epidermal Growth Factor Receptor 2), Glycoprotein (G-Protein) of Rabies Virus, HER2/neu peptide, Vκ₃₋₂₀ (non-Hodgkin's lymphoma light chain idiotype).

The invention is disclosed in more detail through the experimental section here below.

### 1) Effect of Molecular Weight and Conjugation to the Antigen

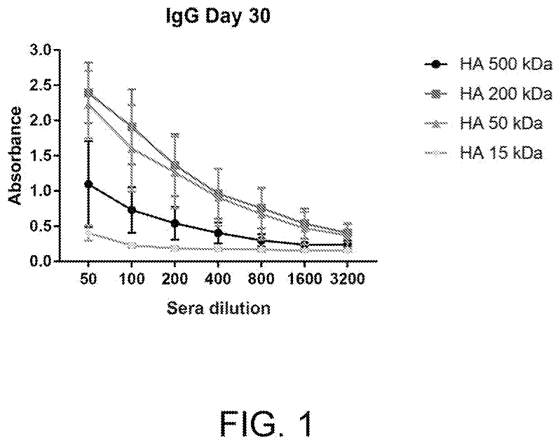

To determine the molecular weight of HA displaying the better adjuvant property, Balb/c mice were immunized by intramuscular injection (i.m.) with different molecular size of HA (500, 200, 50, and 15 kDa) conjugated to ovalbumin (OVA). Animals were vaccinated with the standard immunization schedule, which consists in a priming injection at day 0 and two boosts at day 14 and 21. The relative sera were collected at day 0, before every inoculum and then once per month up to 1 year. Sera were analyzed by enzyme linked immunosorbent assay (ELISA) to determine the antigen-specific total IgG antibody content. ELISA plates were coated with the specific antigen and then incubated with serial dilutions of mice sera. After washing, plates were incubated with the secondary HRP-conjugated anti-mouse IgG antibody. After a single priming injection (analysis performed at day 14), only 200 kDa HA was able to induce an IgG response in mice immunized with 10 μg of OVA, while similar levels of IgG were reached only after two injections of OVA conjugated to 50 kDa HA. Notably also large-sized HA showed to induce a low production of IgG, whereas the 15 kDa HA was less efficient in eliciting an OVA-specific humoral response even after 3 doses, demonstrating that the HA adjuvant effect depends on its molecular size, and can be mostly restricted to a range from 200 to 50 kDa (FIG. 1).

Moreover, to evaluate the role played by chemical conjugation to the antigen in HA adjuvanticity, humoral responses were evaluated in mice immunized (as described above) with OVA extemporaneously mixed with HA, and compared with those induced by HA-Ag bioconjugates and antigen injected alone. Sera were collected at different time points and IgG content analyzed by ELISA test (as described above). In mice immunized with HA simply mixed with Ag, the adjuvant effect was entirely lost. IgG production in mice immunized with HA+Ag was indeed comparable to that obtained after immunization with Ag alone, clearly indicating that the covalent binding of HA to the antigen is required and necessary for HA to exert its adjuvant effect.

### 2) HA-Induced Specific Humoral Responses

Mice were immunized by intramuscular injection (as described in point 1) with different protein antigens or peptides (i.e. OVA, BSA, RNase, H5N1, hGH, TT, HER2/neu, G-Protein, Vκ₃₋₂₀, and HER2/neu peptides) conjugated to HA, and sera were assessed by ELISA as described in section

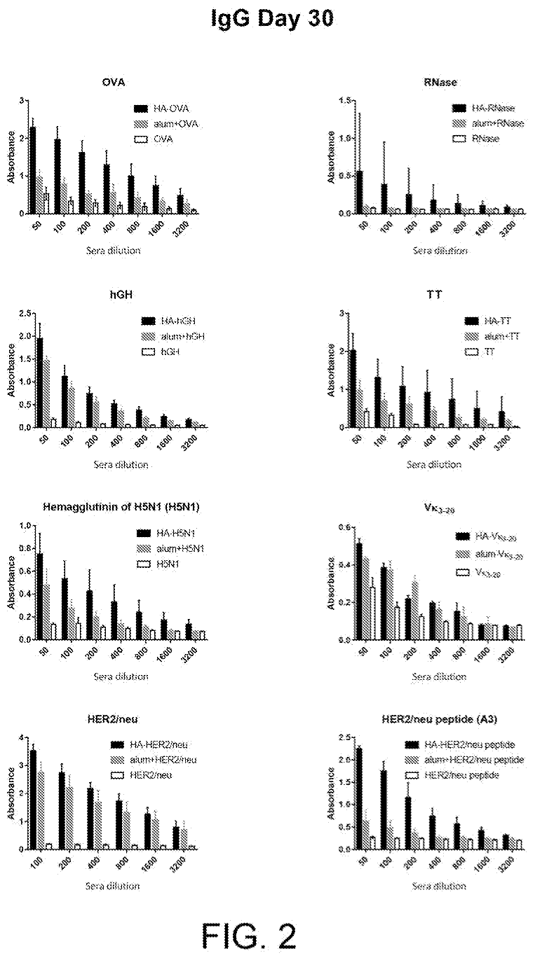

1. Results obtained in immunized mice revealed that HA fosters a strong humoral response that was superior to that obtained with Alum. These results demonstrate that HA acts as a powerful adjuvant capable of fostering strong humoral responses against a wide range of protein antigens and peptides FIG. 2).

### 3) Biocompatibility and Toxicity of HA

To assess the safety profile of HA and evaluate the potential induction of local toxicity, Balb/c mice were injected once i.m. with the model antigen OVA alone, chemically linked to HA, emulsified with Alum, CFA/IFA, Addavax, ISA 51 and ISA 720 or simply mixed with LPS, Chitosan or Quil-A. Animals were sacrificed at different time points (6 hours and 1, 3, 7, and 14 days after immunization) and tibialis anterior muscles (the muscle site of injection) were collected for histological analysis after hematoxylin and eosin (H&E) staining.

HA-injected muscles disclosed a totally preserved and intact tissue texture, without any trace of inflammatory cell infiltration. Histologies derived from mice immunized with HA-OVA were indeed comparable with those obtained following injection of OVA alone. Differently, muscles from mice injected with all the other commercial adjuvants showed clear signs of inflammation, displaying massive recruitment of inflammatory cells at injection site already 24 h after immunization, which still persisted 7 days after injection. In addition, traces of local damage likely caused by the accumulation of oil particles were detected in specimens from mice immunized with oil-in-water emulsion adjuvants such as CFA/IFA and Montanide.

### 4) HA Enhances the Immunogenicity of Conjugated Antigens and Induces Specific, High and Long-Lasting Humoral Responses

To assess the effects of immunization with the HA-OVA bioconjugate in vivo and the levels of antigen-specific humoral response induced by HA, Balb/c mice were injected i.m. (as described in point 1) with the model antigen OVA either unconjugated, conjugated to HA or emulsified with Alum. OVA-specific total IgG, and IgG1, IgG2a, IgG2b subclass levels were measured by ELISA assay and monitored over time as described in point 1.

The protein alone induced only a negligible Ab response, which became evident and detectable upon Alum addition. Conversely, animals immunized with HA adjuvant showed a very robust and strong anti-OVA humoral response, with highly increased IgG production in comparison to Alum. When IgG subclasses were analyzed, HA turned out not only to induce significantly higher IgG1 levels but, differently from Alum, was also able to elicit the production of IgG2a and IgG2b subclasses. Kinetics obtained by monitoring over time the IgG production in sera of immunized mice, showed how the HA-induced humoral response was long-lasting, remaining detectable for 1 year after priming. Notably, also IgG2a and IgG2b Abs were still detectable 1 year after the first injection of HA-OVA bioconjugate.

### 5) HA is Efficient After a Single Dose

With the aim of reducing the number of injections, we applied a single-dose immunization schedule where mice were injected only once with 10 μg of OVA. Thereafter, mice humoral response was monitored for one year to evaluate the kinetics of IgG production (as described in section 1).

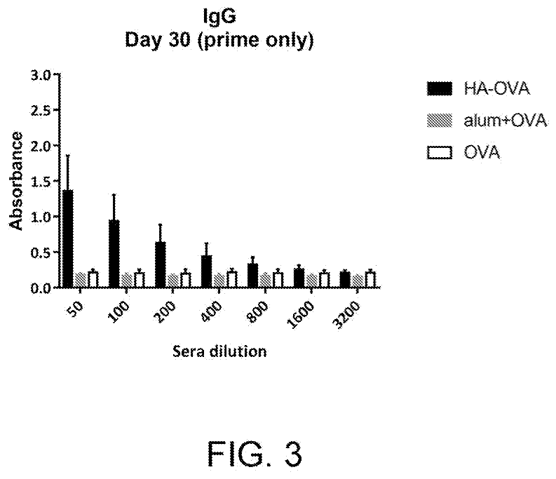

Such long-term follow up disclosed that HA-adjuvanted immunization was able to induce a long-lasting humoral response also with a single dose; this completely diverged from Alum, which showed to be unable to elicit a detectable humoral response after a single inoculation, but required repeated immunization steps (FIG. 3). Further analysis of IgG subclasses performed 30 days after vaccination showed that HA-induced humoral response was mainly due to IgG1, although detectable amounts of IgG2a were observable.

### 6) Immunogenicity of Low Antigen Concentrations is Favored by HA Adjuvanticity

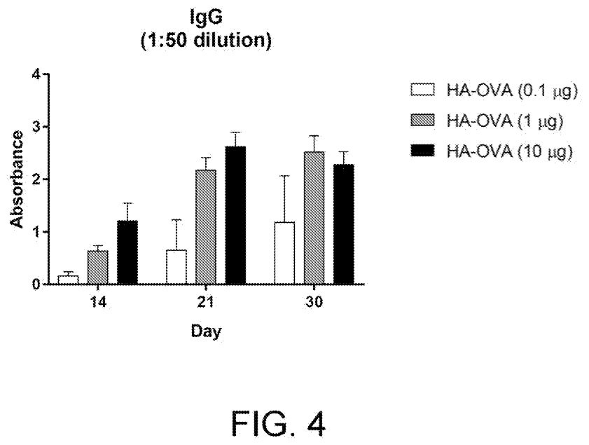

To assess whether HA improves the immunogenicity of low antigen dose, OVA was titrated and injected at the dose of 0.1, 1 or 10 μg in Balb/c mice following the standard schedule, and total IgG production was evaluated (as described in section 1). Results obtained in mice immunized with 0.1 μg of OVA revealed that HA, differently from Alum, succeeded in inducing a detectable production of IgG even with such low antigen dose, and results were already relevant just after the second injection. This IgG production increased with the antigen dose, being clearly detectable at day 14 in animals receiving 1 μg of OVA and reaching, at day 30 with the same dosage, an amount of IgG that was comparable to that obtained with 10 μg of OVA. These results demonstrate that HA acts as a powerful adjuvant capable of fostering strong humoral responses even following immunization with very low antigen concentrations (FIG. 4).

### 7) HA Breaks the Tolerance Against Self-antigens: The Transgenic Balb-neuT Mice Model

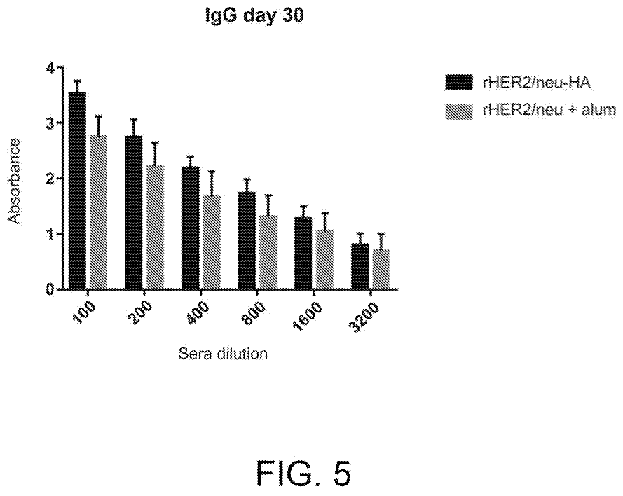

Female Balb-neuT mice are transgenic mice that progressively develop breast tumors in all ten mammary glands that overexpress the activated rat HER2/neu (rHER2/neu) oncogene. To assess if HA adjuvant is able to break the tolerance against the self-antigen rHER2/neu, female Balb-neuT mice were immunized i.m. with 10 μg of rHER2/neu-HA or rHER2/neu emulsified in Alum using the standard protocol (prime+2 boosts) described in point 1). Sera were collected from day 0 to the day of sacrifice at different time points, and IgG and IgG subclasses content (IgG1, IgG2a and IgG2b) were analyzed by ELISA test as described in point 1). Both HA and Alum demonstrated to be able to break the tolerance against the self-antigen rHER2/neu. Nevertheless, HA-vaccinated Balb-neuT mice disclosed higher antigen-specific antibody titers (FIG. 5: example of total IgG titers obtained by ELISA test of day 30 sera) that were already detectable after the priming dose. On the other hand, Alum required at least a boost to induce antigen-specific humoral responses. While both adjuvants elicited almost comparable amounts of IgG1 antibodies, the HA-conjugate stood out for the ability to better induce the production of the two subclasses IgG2a and IgG2b.

### 8) HA is an Adjuvant Suitable for Different Injection Routes (i.e., i.m. and i.v.)

Balb/c mice were immunized by i.m. or intravenous (i.v.) injection of OVA alone (10 μg/mouse) or chemically linked to HA (as described in point 1), while mice receiving the antigen emulsified with Alum were treated only i.m. as Alum cannot be administered i.v.

OVA-specific total IgG, and IgG1, IgG2a, IgG2b subclass amounts were assessed by ELISA assay and monitored over time as described in section 1.

Time course quantification of OVA-specific IgG showed that the humoral response induced by HA-OVA remained detectable for a period of 1 year after priming, following both the i.m. and i.v. injection. Evaluation of IgG subclasses displayed that both i.m. and i.v. injection routes enhanced not only IgG1 subclass but also IgG2a and IgG2b and, notably, also these subclasses were still detectable for a period up to one year after the first injection of HA-OVA bioconjugate. On the other hand, mice receiving OVA alone did not disclosed any humoral response, while antibody titers in mice inoculated with OVA adjuvanted with Alum were always much lower than those detected in HA-immunized animals and represented only by IgG1. Similar data were also obtained after intraperitoneal (i.p.) injection.

Taken together this data indicate that HA is able to induce high and long-lasting humoral responses also when non-conventional (i.e., i.v. or i.p.) administration routes are employed.

9) HA is an Adjuvant Comparable or Even Superior to Other Commercial Adjuvants and is Always More Efficient Than Other Adjuvants Already Approved for Clinical Use (e.g. Alum, Addavax)

Different commercially available adjuvants were selected from the most common adjuvant classes, and compared to HA either using the standard schedule (as described in point 1) or a single injection. Balb/c mice were injected i.m. with the model antigen OVA alone, chemically linked to HA, emulsified with Alum, CFA/IFA, Addavax, ISA 51 and ISA 720 or simply mixed with LPS, Chitosan or Quil-A. OVA-specific total IgG amounts were measured by ELISA assay and monitored over time as described in point 1 above.

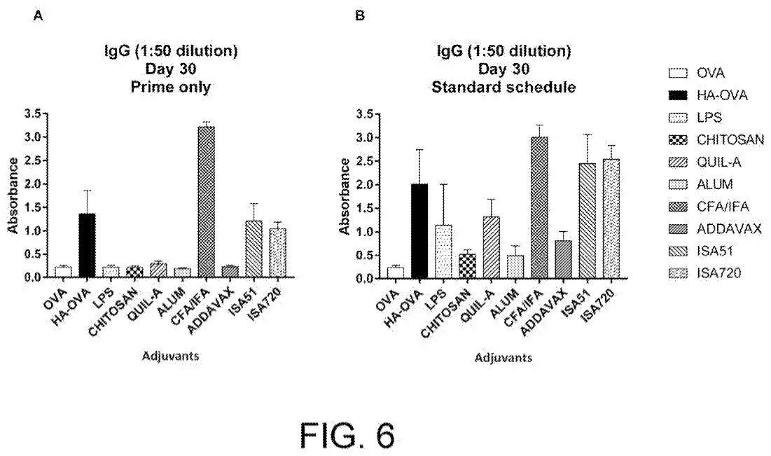

Analysis of humoral responses after a single priming in Balb/c mice, revealed that HA-induced IgG production was only lower than that induced by CFA/IFA adjuvant, and comparable to that produced by Montanide ISA 51 and ISA 720, but resulted always superior to all the other adjuvants tested (FIG. 6). Following immunization with the standard schedule, HA-induced IgG approached the levels reached by CFA/IFA and the two Montanide versions, being however still higher than values achieved with the other adjuvant tested. Therefore, the adjuvant effect of HA is at least comparable to that produced by most of the other experimental adjuvants tested, but largely outperforms the activity of Alum and Addavax, which are the only adjuvants of this group approved for human use.

### 10) HA Induces Antibodies Able to Stimulate Complement-Dependent Cytotoxicity (CDC)

Female Balb/c and Balb-neuT mice were vaccinated with rHER2/neu-HA or rHER2/neu emulsified in Alum using the classical vaccination schedule described in point 1. Sera were collected at day 0, before every inoculum and then once per month up to 1 year (Balb/c mice) or sacrifice day (Balb-neuT mice).

To test the ability of vaccine-induced antibodies to mediate the complement-dependent lysis of rHER2/neu-expressing tumor cells, every single serum sample was incubated with the rHER2/neu-expressing 3T3/NKB cell line and the rHER2/neu-negative NIH/3T3 cell line, which were previously radiolabeled with ⁵¹Cr. After washing, cells were incubated with rabbit complement. Supernatants were evaluated for their radioactivity content following addition of rabbit complement, using a γ-ray counter. The cytotoxicity index was expressed as the percentage of specific lysis.

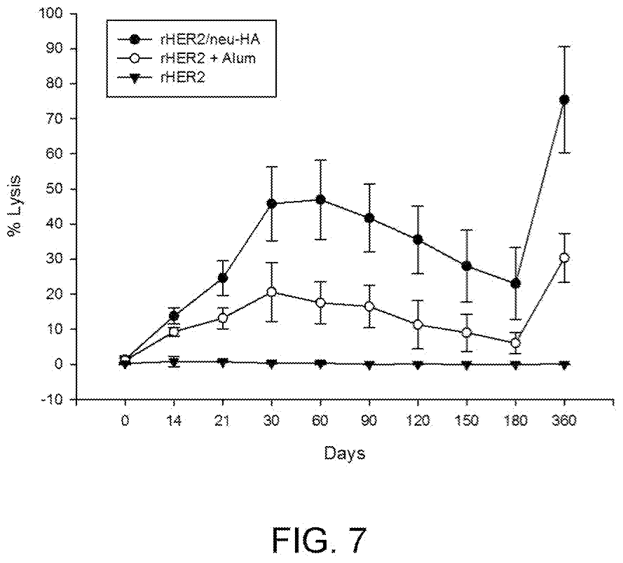

Both HA and Alum adjuvants stimulated in both animal strains the production of immunoglobulins able to mediate cell lysis by activating the complement cascade. CDC was specific for only rHER2/neu expressing cells, since NIH/3T3 cells viability was not affected by the presence of mouse sera and rabbit complement. Of note, the antibodies stimulated in HA-vaccinated mice were more efficient in mediating 3T3/NKB cell lysis, than those obtained with Alum-based vaccination, suggesting that HA is more efficient in inducing effector immune responses. Likely, the difference in mediating CDC may correlates to the differences in the production of IgG subclasses by the two adjuvants. Indeed, HA differs from Alum in stimulating the production of IgG2a and IgG2b subclasses, responsible in mice for mediating CDC. Surprisingly and despite the low IgG content, sera collected 1 year after Balb/c mice vaccination displayed an improved ability to trigger 3T3/NKB lysis (about 90%). This suggest that HA-vaccination seems to stimulate also the “maturation” of antibody-mediated effector responses (FIG. 7).

### 11) HA is Extremely Effective in Tumor Protection

To assess the ability of HA-based vaccination to induce antitumor immunity and then to protect vaccinated subjects from tumor appearance and growth, Balb/c mice were immunized i.m. with 10 or 1 μg of rHER2/neu-HA or rHER2/neu+Alum using the classical schedule reported in point 1. At day 30 after vaccination, mice were challenged with 1×10⁵ TUBO cells injected into the mammary fat pad. Control mice were not vaccinated and only tumor challenged. Mice were observed 3 times per week to monitor and measure tumor growth. Tumor mass was calculated applying the formula: T mass=(minimum diameter)²×(maximum diameter)/2. Mice were sacrificed when tumor masses reached a volume of ≥500 mm³ or when skin ulceration occurred. The experiment was repeated three times.

Ten μg of rHER2/neu-HA completely protected mice from tumor growth, since all the mice did not develop any detectable tumors. Of note, the lower dose consisting in 1 μg of rHER2/neu-HA still demonstrated a remarkable tumor prevention activity since about 70% of mice survived tumor challenge. Contrarily, Alum failed to confer any tumor protection, and mice were sacrificed within 5 weeks, as for the control group.

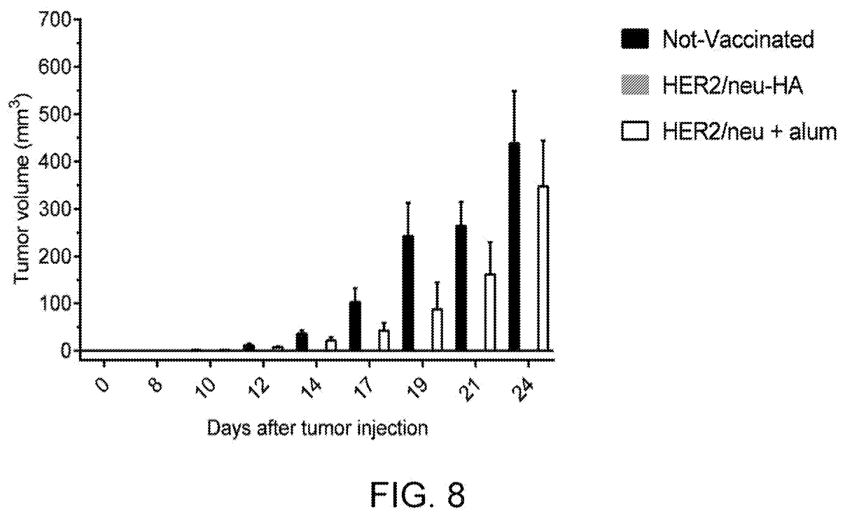

Therefore, HA-based vaccination demonstrated to be extremely effective in tumor prevention, inducing antitumor immune responses (FIG. 8).

### 12) HA is Effective in Treating and Delaying Pre-Neoplastic Lesions in Balb-neuT Mice

The ability of HA-based vaccination to prevent/delay the occurrence of spontaneous tumors in mice bearing pre-neoplastic lesions was assessed by vaccinating Balb-neuT mice, which represent an immunotolerant model of spontaneous and aggressive rHER2/neu-positive cancer. Balb-neuT mice were thus vaccinated with 10 μg of rHER2/neu-HA or rHER2/neu+Alum using the classical immunization schedule (see point 1), and the appearance and growth of spontaneous mammary tumors was monitored. Control group was represented by non-vaccinated Balb-neuT mice. Mice were sacrificed when at least one of the 10 mammary glands developed a tumor that reached a volume of 1500 mm³ or when skin ulceration occurred. The experiment was repeated three times.

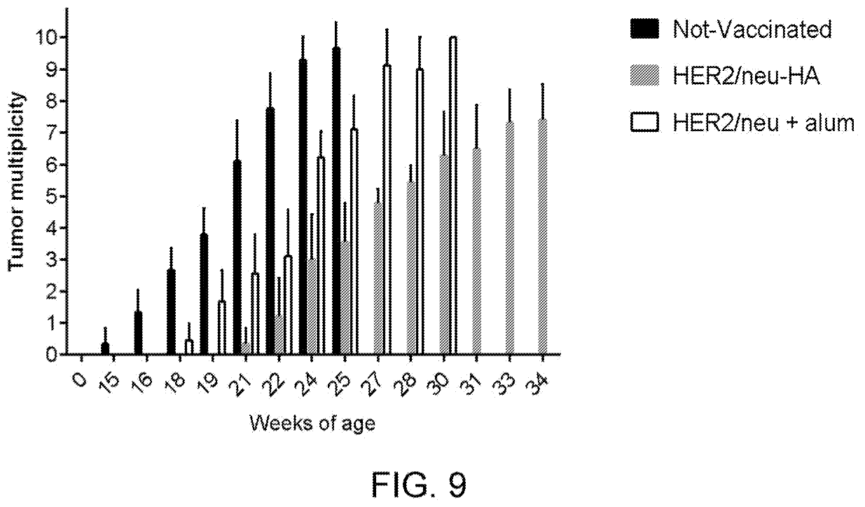

Control groups developed the first tumor by week 16 of age, while at week 25 all 10 mammary glands developed invasive tumors. Despite Alum succeeded in stimulating antibody production in vaccinated mice, these responses did not translate in conferring any tumor protection, since mice were sacrificed contemporarily with control non-vaccinated group. Conversely, HA-vaccinated Balb-neuT mice showed a prolonged tumor-free survival, as the first tumor appeared around week 20. Moreover, tumors appeared to be smaller in volume as compared to Alum-vaccinated and control groups, due to the slowing of tumor growth. In addition, in the HA-vaccinated group there was a decrease in the number of mammary glands involved, as compared to both Alum-vaccinated and non-vaccinated groups (FIG. 9). Therefore, HA-based vaccination demonstrated to be efficient in breaking tolerance against self tumor antigens, stimulating anti-tumor responses that strongly delay and reduce rHER2/neu-overexpressing mammary tumor growth.

13) HA Delays Tumor Growth and/or Promote Tumor Regression in a Therapeutic Setting

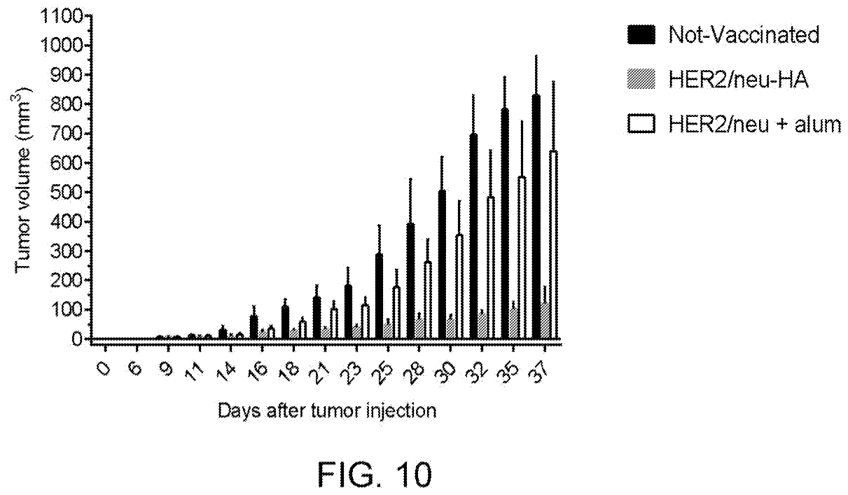

To assess the ability of HA-based vaccination to stimulate anti-tumor immune responses in a therapeutic setting, female Balb/c mice were firstly challenged with 1×10⁵ TUBO cells injected into the mammary fat pad; when all mice had an established tumor (10 days after inoculum, called “day 0 of vaccination”) they were vaccinated i.m. at days 0, 7, and 14 with 10 or 1 μg of rHER2/neu-HA or rHER2/neu emulsified in Alum. Again, control group was represented by not-vaccinated mice. Mice were observed 3 times per week to monitor and measure tumor growth. Tumor mass was calculated as described in point 11. Mice were sacrificed when tumors reached the volume of 1000 mm³ or when skin ulceration occurred. The experiment was repeated three times. Even though both adjuvants demonstrated to stimulate antitumor responses, Alum-vaccinated group benefited of only a weak delay in tumor growth and in a weak survival improvement, while HA-based vaccination efficiently delayed tumor growth and strongly prolonged survival (FIG. 10). Thus, HA-based vaccination is extremely efficient also in the therapeutic setting, and elicits strong antitumor immune responses able to delay tumor growth or promote complete tumor regression.

### 14) HA Induces not Only a Humoral but Also a Cell-Mediated Immune Response

To assess whether HA vaccination acts not only on the humoral arm of immune system but is also capable of fostering cell-mediated immune responses, mice reported in points 11-13 were further tested for the generation of cytotoxic T cell (CTL) immunity. To this end, spleens were harvested at sacrifice day, and mixed leukocyte tumor cell cultures (MLTC) were set up by restimulating splenocytes in vitro with irradiated rHER2/neu-positive TUBO and 3T3/NKB, or rHER2/neu-negative NIH/3T3 cell lines. After 5 days, CTL in the cultures (called effectors cells or E) were incubated with ⁵¹Cr-labeled TUBO, 3T3/NKB, and NIH/3T3 cells (called target cells or T) using different E/T ratios, to test their ability to recognize and kill only rHER2/neu-expressing tumor cells. After a 4 h-incubation at 37° C., the radioactivity in the supernatants indicating the presence of ⁵¹Cr released by target cells, was detected by a γ-ray counter. The cytotoxicity index was expressed as the percentage of specific lysis at the E/T ratio of 100:1.

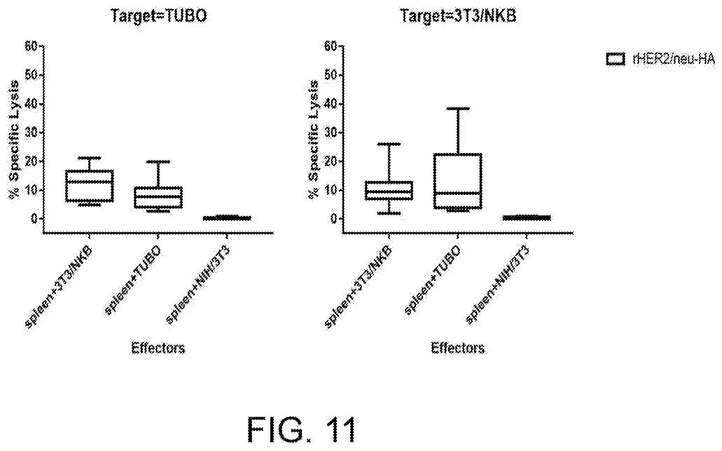

Mice vaccinated with Alum did not mount any CTL response; in contrast, a strong lytic activity was measured in both Balb/c and Balb-neuT mice vaccinated with HA (FIG. 11: percentage of cell lysis obtained for Balb-neuT mice vaccinated with rHER2/neu-HA). In conclusion, HA is extremely efficient in stimulating also the cellular immune compartment.

In view of the above disclosure, there will be evident to the person skilled in the art the advantages offered by the present invention.

First of all, it has been demonstrated that thanks to the hyaluronic acid polymers here disclosed, conjugates can be prepared which are useful alternative to the already known adjuvants.

In particular, they are endowed with a comparable or even superior activity versus many other clinical grade adjuvants.

In addition, thanks to its very high biocompatibility and safety, no inflammation process or effect is seen at the injection site.

Also, it has been observed that the use of hyaluronic acid as the adjuvant induces a more balanced Th1/Th2 response compared to other known adjuvants, first of all the Alum.

As a further advantage, it has been seen that hyaluronic acid is capable of breaking the tolerance against self-antigens.

As a consequence, the immunity system is capable of recognizing the expression or the overexpression of self-antigens caused by a tumor and to trigger a response against that.

The conjugates prepared according to the present invention are stable and extremely water-soluble.

Thus, they can be lyophilized or frozen for long-term storage without losing their potency, and therefore HA-based vaccines are suitable for pandemics or in developing countries.

The experiments performed have shown that the disclosed conjugates can be administered through other routes, like intravenously (i.v.), intramuscularly, intraperitoneally (i.p.), subcutaneously, intranasally, intravitreally or rectally, beside the classical intramuscular (i.m.) administration.

1.-32. (canceled)

33. A prophylactic or therapeutic method, comprising vaccinating a patient with hyaluronic acid.

34. The prophylactic or therapeutic method according to claim 33, wherein hyaluronic acid is an immunological adjuvant.

35. The prophylactic or therapeutic method according to claim 33, wherein hyaluronic acid has a molecular weight ranging from about 10 to 500 KDa.

36. The prophylactic or therapeutic method according to claim 33, wherein hyaluronic acid is conjugated with an antigen.

37. The prophylactic or therapeutic method according to claim 33, wherein hyaluronic acid is chemically bound to an antigen.

38. The prophylactic or therapeutic method according to claim 37, wherein said antigen is a protein-based or a peptide-based antigen.

39. The prophylactic or therapeutic method according to claim 37, wherein said antigen is selected from the group consisting of: OVA (ovalbumin), BSA (Bovine Serum Albumin), RNAse, H5N1 (Influenzae virus), hGH (human Growth Hormone), TT (Tetanus Toxoid), HER2/neu (Human Epidermal GrowthFactorReceptor2), Glycoprotein (G-Protein) of Rabies Virus, HER2/neu peptide, and Vκ₃₋₂₀ (non-Hodgkin's lymphoma light chain idiotype).

40. The prophylactic or therapeutic method according to claim 37, wherein vaccinating comprises a first administration of a hyaluronic acid conjugate.

41. The prophylactic or therapeutic method according to claim 40, wherein said first administration is followed by a second or one or more further administration/s.

42. The prophylactic or therapeutic method according to claim 37, wherein hyaluronic acid conjugates are administered intravenously, intramuscularly, intraperitoneally, subcutaneously, intranasally, intravitreally or rectally.

43. The prophylactic or therapeutic method according to claim 37, wherein the total production of IgG is enhanced.

44. The prophylactic or therapeutic method according to claim 37, wherein the production of IgG subclasses is enhanced.

45. The prophylactic or therapeutic method according to claim 33, wherein hyaluronic acid fosters a cell-mediated immune response and enables generating specific cytotoxic T-cell immunity.

46. The prophylactic or therapeutic method according to claim 37, wherein the IgG are detectable after one year after the first inoculation.

47. The prophylactic or therapeutic method according to claim 33, wherein a single administration of hyaluronic acid is performed.

48. The prophylactic or therapeutic method according to claim 47, wherein a first, a second, and/or one or more further administrations of hyaluronic acid are performed.

49. The prophylactic or therapeutic method according to claim 33, wherein hyaluronic acid is capable of enhancing immunogenicity of an antigen.

50. The prophylactic or therapeutic method according to claim 33, whereinthe antigen is a self-antigen.

51. The prophylactic or therapeutic method according to claim 50, wherein the self-antigen is over-expressed.

52. The prophylactic or therapeutic method according to claim 51, wherein the self-antigen is mutated.

53. The prophylactic or therapeutic method according to claim 51, wherein the self-antigen is mutated and over-expressed.

54. The prophylactic or therapeutic method according to claim 51, wherein the self-antigen is afetal self-antigen.

55. The prophylactic or therapeutic method according to claim 51, wherein the self-antigen is a cancer testis antigen.

56. The prophylactic or therapeutic method according to claim 33, wherein hyaluronic acid breaks immunologic tolerance against a self-antigen.

57. The prophylactic or therapeutic method according to claim 33, wherein the prophylactic or therapeutic method comprises delaying development and/or growth of pre-neoplastic lesions.

58. The prophylactic or therapeutic method according to claim 33, wherein the prophylactic or therapeutic method comprises delaying development and/or growth of an already existing neoplasia.

59. The prophylactic or therapeutic method according to claim 33, wherein the prophylactic or therapeutic method comprises regressing of an already existing neoplasia.

60. A conjugate of hyaluronic acid and an antigen, wherein hyaluronic acid has a molecular weight of from about 10 to 500 KDa.

61. The conjugate according to claim 60, wherein the hyaluronic acid and the antigen are chemically bound.

62. The conjugate according to claim 60, wherein the antigen is a protein-based or a peptide-base antigen.

63. The conjugate according to claim 61, wherein the antigen is selected from the group consisting of: OVA (ovalbumin), BSA (Bovine Serum Albumin), RNAse, H5N1 (Influenzae virus), hGH (human Growth Hormone), TT (Tetanus Toxoid), HER2/neu (Human Epidermal Growth Factor Receptor 2), Glycoprotein (G-Protein) of Rabies Virus, HER2/neu peptide, and Vκ₃₋₂₀ (non-Hodgkin's lymphoma light chain idiotype).
